(19) United States
(12) Patent Application Publication
(10) Pub. No.: US 2005/0049213 A1
(43) Pub. Date: Mar. 3, 2005

(54) Method for preventing or reversing asthma and compositions useful therefor

(76) Inventor: Devendra K Agrawal, Omaha, NE (US)

Correspondence Address: Peter Rogalskyj;Rogalskyj & Weyand, P O Box 44, Livonia, NY, 14487-0044, US

(21) Appl. No.: 10/492,764

(22) PCT Filed: Oct. 19, 2002
(86) PCT No.: PCT/US02/33562

Related U.S. Application Data
(60) Provisional application No. 60/344,880, filed on Oct. 19, 2001.

(51) Int. Cl. A61K 48/00; A61K 38/18

(52) U.S. Cl. 514/44; 514/2; 424/93.2

(57) ABSTRACT

Disclosed are methods for partially or completely preventing or reversing the effects of asthma in a subject. The methods include administering an effective amount of a Flt3 ligand to the subject Compositions which include a Flt3 ligand and a pharmaceutically acceptable aerosolizing agent are also described, as are conjugates which include a Flt3 ligand and an allergen.

This application claims the benefit of U.S. Provisional Patent Application Ser. No. 60/344,880, filed Oct. 19, 2001, which is hereby incorporated by reference.

### FIELD OF THE INVENTION

The subject invention is directed generally to methods for partially or completely preventing or reversing the effects of asthma in a subject and to compositions that are useful in such methods.

### BACKGROUND OF THE INVENTION

More than ten million persons in the United States suffer from asthma and related inflammatory lung diseases. The numbers of persons with asthma is increasing both in the United States and worldwide. The morbidity associated with asthma makes asthma a major medical condition. Asthma is the most common chronic disease of childhood and the leading cause among chronic illnesses of school absences. Asthma in humans results in an estimated 27 million patient visits, 6 million lost workdays, and 90.5 million days of restricted activity per year. In addition to its morbidity, the mortality rate for asthma is growing worldwide. Additionally, asthma reactions are a growing problem for animals. In particular, the horse racing industry is affected by horses that suffer from asthmatic reactions.

Asthma is a lung disease characterized by a usually reversible airway obstruction, airway inflammation and increased airway responsiveness to stimuli. The airway obstruction in an asthma attack is thought to be due to the combination of bronchospasm of the smooth muscles of the bronchial tree, increased mucous secretion, edema of airway mucosa due to increased vascular permeability, cellular infiltration of the airway walls, and injury to airway epithelium.

Asthma may be triggered by a variety of causes such as allergic reactions, a secondary response to infections, industrial or occupational exposures, ingestion of certain chemicals or drugs, exercise, and vasculitis. Regardless of the trigger, many of the pathological features of asthma can be attributed to mast cell degranulation. Mast cells will degranulate in response to many conditions in addition to the classical IgE-antigen stimulation. Not wishing to be bound by the following theory, it is theorized that when the asthmatic, human or animal, inhales an allergenic substance, sensitized IgE antibodies trigger mast cell degranulation in the lung interstitium. The mast cell degranulation releases among other factors, histamine, bradykinin, and slow-reacting substance of anaphylaxis (“SRS-A”) which includes the leukotrienes C, D and E, prostaglandins including PGF₂, PGF₂α, and PGD₂, and thromboxane A₂. The histamine then attaches to receptor sites in the larger bronchi, causing irritation, inflammation and edema. The SRS-A attaches to receptor sites in the smaller bronchi, causing edema and attracting prostaglandins, which enhance the effects of histamine in the lungs. With the help of the prostaglandins, histamine also stimulates excessive mucous secretion, further narrowing the bronchial lumen. When the asthmatic inhales, the narrowed bronchial lumen still expands slightly, allowing air to reach the alveoli. However, upon exertion to exhale, the increased thoracic pressure closes the bronchial lumen completely. Thus, in an asthma attack, air can enter, but not exit the lungs. Mucous then fills the lung bases, inhibiting alveolar ventilation. In an effort to compensate for lowered alveolar ventilation, blood is shunted to other alveoli. Without adequate compensation, hypoxia, and in extreme cases, respiratory acidosis may result.

In many cases, there are two phases to an allergic asthma attack, an early phase and a late phase which follows 4-6 hours after bronchial stimulation. The early phase includes the immediate inflammatory response including the reactions caused by the release of cellular mediators from mast cells. Late phase reactions develop over a period of hours and are characterized histologically by an early influx of polymorphonuclear leukocytes and fibrin deposition followed later by infiltration of eosinophils. Late phase reactions increase airway reactivity and lead to prolonged asthmatic exacerbations that may last from hours to days to months in some subjects. One of the residual effects of asthma reactions is this hyperresponsiveness of the airways to nonspecific stimuli.

The current treatments for asthma are not adequate and many have serious side effects. The general goals of drug therapy for asthma are prevention of bronchospasm and control of airway hyperreactivity or hyperresponsiveness, an indication of airway inflammation. One effective treatment is avoidance of all allergens that trigger an asthma attack. Though scrupulous housecleaning and air cleansing devices can lessen the exposures to allergens, it is very difficult to eliminate all exposures to allergens. Thus, most asthmatics are treated with pharmacological agents that have side effects.

Another common treatment regimen is administration of adrenergic agonists. These compounds mimic the physiological effects of the adrenal medullary hormones and neurotransmitters of the sympathetic nervous system. It is believed that adrenergic agonists operate by stimulating the β₂-receptors in the lung, which, in turn cause smooth muscle relaxation, increased chloride fluxes, and reduced vascular permeability. However, many side effects result from treatment with adrenergic agonists because the adrenergic agonists are generally not selective for only the β₂-receptors, but also effect the α-receptors and β₁-receptors. β₁-Receptors are found in the heart, and adrenergic stimulation also leads to cardiac stimulation, which is a serious side effect of treatment with adrenergic agonists. Additionally, many of these compounds are rapidly metabolized and have very short half-lives and, thus, are not effective therapy for asthma or hyperresponsiveness reactions. β₂-adrenergic agonists can be used for treatment of bronchospasm, but have no effect on airway inflammation or bronchial hyperreactivity. In fact, chronic use of β₂-adrenergic agents alone, by down regulation of β₂-receptors, may worsen bronchial hyperreactivity.

Asthma can also be treated with methylxanthines, such as theophylline. There is substantial variability in the absorbance and clearance of theophylline among animals. Even in individuals, theophylline clearance is affected by many physiological situations, such as infection, antibiotic use, cigarette use, and diet. The side effects of theophylline are nervousness, nausea, vomiting, anorexia, abdominal discomfort, and headache. It is difficult to reach an effective drug level that provides asthma control without triggering side effects.

Corticosteroids are used to treat asthma by reducing the inflammatory component. Because the latephase asthmatic response is poorly responsive to bronchodilators, corticosteroids are used to treat late-phase and airway hyperreactivity reactions. These agents have high toxicity in children, including adrenal suppression and reduced bone density and growth. In all age groups, corticosteroids have numerous side effects and complications which impact major organ systems. Use of oral corticosteroids must be closely monitored, and its use curtailed or halted as soon as possible.

Cromolyn, another well known asthma therapeutic, acts by stabilizing mast cells and reducing or preventing release of the cellular mediators. Thus, cromolyn is effective in stopping or reducing both the early and late phases of asthma inflammatory reactions. However, cromolyn is only effective in preventing the onset of an asthma reaction if given prior to an asthma attack. Once the asthma reaction has begun, the mediators have been released and treatment with cromolyn would do nothing to relieve the bronchoconstriction and hyperresponsiveness. Thus, asthma patients would have to take cromolyn continuously to prevent future asthma attacks that may or may not occur.

In view of the above and for other reasons, a need continues to exist for methods and compositions for partially or completely preventing or reversing the effects of asthma. The present invention is, in part, directed to such a need.

### SUMMARY OF THE INVENTION

The present invention relates to a method of partially or completely preventing or reversing the effects of asthma in a subject. The method includes administering an effective amount of a Flt3 ligand to the subject.

The present invention also relates to a composition which includes a Flt3 ligand and an pharmaceutically acceptable aerosolizing agent.

The present invention also relates to a conjugate which includes a Flt3 ligand and an allergen.

### BRIEF DESCRIPTION OF THE DRAWINGS

FIGS. 1A-1B are graphs showing the effect of Flt3 ligand administration on allergic response.

FIGS. 2A-2B are graphs showing the effect of Flt3 ligand administration on allergic response.

FIG. 3 is a graph showing the effect of Flt3 ligand administration on airway hyperresponsiveness to methacholine.

FIGS. 4A-4C are graphs showing the effect of Flt3 ligand administration on reversing allergic response and airway hyperresponsiveness to methacholine.

FIGS. 5A-5D are graphs showing the effect of Flt3 ligand administration on serum cytokines and serum total IgE levels.

FIGS. 6A-6I are images showing the effect of Flt3 ligand administration on lung histology.

FIGS. 7A-7I are images showing the effect of Flt3 ligand administration on goblet cell hyperplasia and mucus hypersecretion in the lower airways.

FIGS. 8A and 8B are graphs showing the effect of Flt3 ligand administration on reversing airway hyperresponsiveness to methacholine in unrestrained and in tracheostomized mice.

FIGS. 9A-9F are flow cytometry scatter plots showing the effect of Flt3 ligand administration on the fluorescence characteristics of lung dendritic cells labeled with FITC-conjugated CD8α or CD11b antibody and PE-conjugated CD11c+ antibody.

FIGS. 10A-D are graphs showing the effect of Flt3 ligand administration on TNFα, IL-2, IL-4, and IL-5 production.

FIG. 11A is a graphic which depicts a timeline which was used in connection with experiments conducted to determine the effect of a Flt3 ligand gene-containing plasmid on airway hyperresponsiveness and cell infiltration. FIG. 11B is a graph showing the effect of administering a Flt3 ligand DNA molecule-containing plasmid on airway hyperresponsiveness. FIG. 11C is a graph showing the effect of administering a Flt3 ligand DNA molecule-containing plasmid on the presence of bronchoalveolar lavage cells. FIG. 11D is a graph showing the effect of administering a Flt3 ligand and of administering a Flt3 ligand DNA molecule-containing plasmid on differential cells of bronchoalveolar lavage fluid.

### DETAILED DESCRIPTION OF THE INVENTION

The present invention relates to a method of partially or completely preventing or reversing the effects of asthma in a subject. The method includes administering, to the subject, an effective amount of a Flt3 ligand.

As used herein, “asthma” is meant to refer to a disorder of the respiratory system characterized by inflammation, narrowing of the airways, and/or increased reactivity of the airways to inhaled agents. Asthma is frequently, although not exclusively, associated with atopic or allergic symptoms. The effects of asthma which are reversed or prevented by the method of the present invention include, for example, airway hyperresponsiveness to one or more environmental or other allergens, airway inflammation, airway obstruction, tissue and/or blood eosinophilia, and/or mucus hypersecretion. The effects of asthma can be evaluated clinically, cellularly, serologically, or by any other suitable method, some of which are discussed in the Examples which follow.

Illustratively, the asthmatic response can, in some cases, be characterized as a type I hypersensitivity reaction. This can involve allergen-specific immunoglobulins of the IgE class bound to high-affinity receptors on the surfaces of mast cells present in the sub-epithelial layer of the airways. Cross-linking of these bound IgE molecules results in an immediate release of mediators, including leukotrienes, prostaglandins and histamine, which are capable of contracting airway smooth muscle cells and induce edema and mucus secretion leading to narrowed, spastic airways. Although airborne allergens are common triggers of these attacks in allergic asthmatics, other agents (such as cold air, lower respiratory tract infections, and stress) can also stimulate attacks. In addition to the immediate release of bronchospastic mediators, cytokines and chemokines can be locally produced. Chemokines stimulate the recruitment of eosinophils, macrophages, neutrophils, and T lymphocytes. Once present, effector cells, such as eosinophils, may be prompted to release a collection of toxic granules. These granules may cause further, prolonged bronchoconstriction and damage epithelial layers. This damage, coupled with profibrotic cytokines also released by eosinophils and epithelial cells, can lay the groundwork for the process of airway remodeling to begin. Further, cytokines released at the time of mast cell degranulation can have more global effects. These include the recruitment of eosinophils from bone marrow and peripheral sources in addition to encouraging their survival (primarily via IL-5 and GM-CSF) and the stimulation and continued production of IgE by B-cells as well as the induction of vascular cell adhesion molecule-1 (“VCAM-1”) by endothelial cells (IL-4). Moreover, cytokines, such as IL-4 and IL-5, can have the effect of ensuring that this cycle of allergic inflammation persists. The method of the present invention can be used to prevent or reverse some or all of these effects of asthma.

“Partial” reversal and “partial” prevention, as used herein, are meant to refer to any measurable decrease in the effects of asthma, such as, for example, a decrease of at least about 5%, a decrease of at least about 10%, a decrease of at least about 20%, a decrease of at least about 30%, a decrease of at least about 50%, a decrease of at least about 75%, etc. “Complete” reversal and “complete” prevention, as used herein, are meant to refer to the absence, within experimental error, of any measurable effect of asthma, for example, as compared with a non-asthmatic control.

As used herein, “Flt3 ligand” refers to a compound which binds to a cell surface tyrosine kinase, flt3 receptor and is meant to include any member of the class of compounds described in EP 0627487 A2, in WO 94/28391, in U.S. Pat. No. 5,554,512 to Lyman et al., in U.S. Pat. No. 5,554,512 to Lyman et al., in Lyman et al., “Cloning of the Human Homologue of the Murine Flt3 Ligand: A Growth Factor for Early Hematopoietic Progenitor Cells,” Blood, 83:2795-2801 (1994), and/or U.S. Pat. No. 6,291,661 to Graddis et al., each of which is hereby incorporated by reference. The Flt3 ligand can be one which is produced by synthetic peptide chemistry, or it can be recombinant (e.g., produced by expression of a nucleic acid molecule encoding the Flt3 ligand). Illustratively, “Flt3 ligand” is meant to include polypeptides encoded by the human Flt3 ligand cDNA which is on deposit with the American Tissue Type Culture Collection, Rockville, Md., under accession number ATCC 69382, or an appropriate portion thereof.

The Flt3 ligand can be administered in the form of a conjugate which includes the Flt3 ligand and an allergen. An “allergen”, as used herein, refers to a substance (antigen) that can induce an allergic or asthmatic response in a susceptible subject. Suitable allergens for use in such conjugates include, for example, pollens, insect venoms, animal dander, dust, fungal spores, and drugs (e.g. penicillin). Other allergens for use in such conjugates include, for example, ovalbumin, house dust mite, cockroach, and Schistosoma mansoni. Still further examples of natural, animal, and plant allergens include proteins specific to the following genuses: Canine (Canis familiaris); Dermatophagoides (e.g. Dermatophagoides farinae); Felis (Felis domesticus); Ambrosia (Ambrosia artemiisfolia; Lolium (e.g. Lolium perenne or Lolium multiflorum); Cryptomeria (Cryptomeria japonica); Alternaria (Alternaria alternata); Alder; Alnus (Alnus gultinoasa); Betula (Betula verrucosa); Quercus (Quercus alba); Olea (Olea europa); Artemisia (Artemisia vulgaris); Plantago (e.g. Plantago lanceolata); Parietaria (e.g. Parietaria officinalis or Parietaria judaica); Blattella (e.g. Blattella germanica); Apis (e.g. Apis multiflorum); Cupressus (e.g. Cupressus sempervirens, Cupressus arizonica and Cupressus macrocarpa); Juniperus (e.g. Juniperus sabinoides, Juniperus virginiana, Juniperus communis and Juniperus ashei); Thuya (e.g. Thuya orientalis); Chamaecyparis (e.g. Chamaecyparis obtusa); Periplaneta (e.g. Periplaneta americana); Agropyron (e.g. Agropyron repens); Secale (e.g. Secale cereale); Triticum (e.g. Triticum aestivum); Dactylis (e.g. Dactylis glomerata); Festuca (e.g. Festuca elatior); Poa (e.g. Poa pratensis or Poa compressa); Avena (e.g. Avena sativa); Holcus (e.g. Holcus lanatus); Anthoxanthum (e.g. Anthoxanthum odoratum); Arrhenatherum (e.g. Arrhenatherum elatius); Agrostis (e.g. Agrostis alba); Phleum (e.g. Phleum pratense); Phalaris (e.g. Phalaris arundinacea); Paspalum (e.g. Paspalum notatum); Sorghum (e.g. Sorghum halepensis); and Bromus (e.g. Bromus inermis). The nature of the coupling between the Flt3 ligand and the allergen is not particularly critical. For example the he Flt3 ligand and the allergen can be coupled to one another by a covalent bond, a hydrogen bond, and/or an ionic bond. The conjugate can be prepared by any conventional method for coupling polypeptides, such as those described in, for example, U.S. Pat. No. 4,946,945 to Wojdani; Youle et al., “Anti-Thy 1.2 Monoclonal Antibody Linked to Ricin Is a Potent Cell-type-specific Toxin, Proc. Natl. Acad. Sci. USA, 77(9):5483-5486 (1980); Kitagawa et al., “Enzyme Immunoassay of Blasticidin S with High Sensitivity: A New and Convenient Method for Preparation of Immunogenic (Hapten-protein) Conjugates,” J. Biochem. 92(2):585-590 (1982); Freytag et al., “A Highly Sensitive Affinity-column-mediated Immunometric Assay, as Exemplified by Digoxin” Clin Chem.,30(3):417-420 (1984), which are herein incorporated by reference. For example, the conjugation between Flt3 ligand and allergens can be carried out by mixing thiolated Flt3 ligand with maleimide-activated allergen at 4° C. overnight, and unconjugated Flt3 ligand can be removed by dialysis against PBS.

The Flt3 ligand can be administered by administering, to the subject, a nucleic acid molecule encoding a Flt3 ligand under conditions effective to permit the nucleic acid molecule to express Flt3 ligand in the subject. Any suitable technique and vector system can be used to introduce and cause expression of the Flt3 ligand in the subject. Illustratively, techniques for introducing the nucleic acid molecules into the host cells may involve the use of expression vectors which comprise the nucleic acid molecules. These expression vectors (such as plasmids and viruses; viruses including bacteriophage) can then be used to introduce the nucleic acid molecules into suitable host cells. For example, DNA encoding the Flt3 ligand can be injected into the nucleus of a host cell or transformed into the host cell using a suitable vector, or mRNA encoding the Flt3 ligand can be injected directly into the host cell, in order to obtain expression of Flt3 ligand in the host cell.

Various methods are known in the art for introducing nucleic acid molecules into host cells. One method is microinjection, in which DNA is injected directly into the nucleus of cells through fine glass needles (or RNA is injected directly into the cytoplasm of cells). Alternatively, DNA can be incubated with an inert carbohydrate polymer (dextran) to which a positively charged chemical group (DEAE, for diethylaminoethyl) has been coupled. The DNA sticks to the DEAE-dextran via its negatively charged phosphate groups. These large DNA-containing particles stick in turn to the surfaces of cells, which are thought to take them in by a process known as endocytosis. Some of the DNA evades destruction in the cytoplasm of the cell and escapes to the nucleus, where it can be transcribed into RNA like any other gene in the cell. In another method, cells efficiently take in DNA in the form of a precipitate with calcium phosphate. In electroporation, cells are placed in a solution containing DNA and subjected to a brief electrical pulse that causes holes to open transiently in their membranes. DNA enters through the holes directly into the cytoplasm, bypassing the endocytotic vesicles through which they pass in the DEAE-dextran and calcium phosphate procedures. DNA can also be incorporated into artificial lipid vesicles, liposomes, which fuse with the cell membrane, delivering their contents directly into the cytoplasm. In an even more direct approach, DNA is absorbed to the surface of tungsten microprojectiles and fired into cells with a device resembling a shotgun.

Further methods for introducing nucleic acid molecules into cells involve the use of viral vectors. One such virus widely used for protein production is an insect virus, baculovirus. For a review of baculovirus vectors, see Miller, “Insect Baculoviruses: Powerful Gene Expression Vectors,” Bioessavs 11(4):91-95 (1989), which is hereby incorporated by reference. Various other viral vectors, such as bacteriophage, vaccinia virus, adenovirus, and retrovirus, can also be used to transform mammalian cells.

As indicated, some of these methods of transforming a cell require the use of an intermediate plasmid vector. U.S. Pat. No. 4,237,224 to Cohen et al., which is hereby incorporated by reference, describes the production of expression systems in the form of recombinant plasmids using restriction enzyme cleavage and ligation with DNA ligase. These recombinant plasmids are then introduced by means of transformation and replicated in unicellular cultures including procaryotic organisms and eucaryotic cells grown in tissue culture. The DNA sequences are cloned into the plasmid vector using standard cloning procedures known in the art, as described, for example, in Sambrook et al., Molecular Cloning: A Laboratory Manual, 2nd Edition, Cold Spring Harbor Laboratory Press, Cold Spring Harbor, N.Y. (1989), which is hereby incorporated by reference.

Several suitable plasmids containing nucleic acid molecules encoding Flt3 ligand that are suitable for use in the practice of this aspect of the method of the present invention are commercially available from Vector Core at the University of Michigan (Ann Arbor, Mich.). These include pUMVC3-hFLex, pUMVC3-mFL, and pUMVC3-mFLex, and the maps for these plasmids are available at http://www.med.umich.edu/vcore/index.html, which is hereby incorporated by reference.

The Flt3 ligand, Flt3 ligand/allergen conjugate, or Flt3 ligand-encoding nucleic acid molecule can be administered alone or in combination with compatible carriers as a composition. Compatible carriers include suitable pharmaceutical carriers or diluents. The diluent or carrier ingredients should be selected so that they do not diminish the effects of the Flt3 ligand used in the present invention.

The compositions herein may be made up in any suitable form appropriate for the desired use. Examples of suitable dosage forms include oral, intranasal, intratracheal, parenteral, and topical dosage forms.

Suitable dosage forms for oral use include tablets, dispersible powders, granules, capsules, suspensions, syrups, and elixirs. Inert diluents and carriers for tablets include, for example, calcium carbonate, sodium carbonate, lactose, and talc. Tablets may also contain granulating and disintegrating agents, such as starch and alginic acid; binding agents, such as starch, gelatin, and acacia; and lubricating agents, such as magnesium stearate, stearic acid, and talc. Tablets may be uncoated or may be coated by known techniques to delay disintegration and absorption. Inert diluents and carriers which may be used in capsules include, for example, calcium carbonate, calcium phosphate, and kaolin. Suspensions, syrups, and elixirs may contain conventional excipients, for example, methyl cellulose, tragacanth, sodium alginate; wetting agents, such as lecithin and polyoxyethylene stearate; and preservatives, such as ethyl-p-hydroxybenzoate.

Dosage forms suitable for parenteral administration include solutions, suspensions, dispersions, emulsions, and the like. They may also be manufactured in the form of sterile solid compositions which can be dissolved or suspended in sterile injectable medium immediately before use. They may contain suspending or dispersing agents known in the art. Examples of parenteral administration are intraventricular, intracerebral, intramuscular, intravenous, intraperitoneal, rectal, and subcutaneous administration.

Intranasal administration of the Flt3 ligand can be carried out via a liquid spray or via an aerosol. For administration to the respiratory tract by inhalation, the Flt3 ligand can be delivered from an insufflator, nebulizer, or a pressurized pack or other convenient means of delivering an aerosol spray. Pressurized packs may comprise a suitable propellant such as dichlorodifluoromethane, trichlorofluoromethane, dichlorotetrafluoroethane, carbon dioxide, or other suitable gas. In the case of a pressurized aerosol, the dosage unit may be determined by providing a valve to deliver a metered amount. Alternatively, for administration by inhalation or insufflation, the Flt3 ligand can be in the form of a dry powder composition, for example, a powder mix of the Flt3 ligand and a suitable powder base such as lactose or starch. The powder composition can be presented in unit dosage form in, for example, capsules, cartridges, or gelatin or blister packs, from which the powder may be administered with the aid of an inhalator or insufflator. For intranasal administration, the Flt3 ligand can be administered via a liquid spray, such as via a plastic bottle atomizer. Typical of these are the Mistometer (Wintrop) and the Medihaler (Riker).

In addition to the above, generally non-active components of the above-described formulations, these formulations can include other active materials, particularly, actives which have been identified as useful in the treatment of asthma, in the alleviation of symptoms associated with asthma, or in the delivery of materials to the lungs (e.g., bronchodilators). These actives can be broad-based agents, such that they also are useful in treating conditions in addition to asthma, or they can be more specific, for example, in the case where the other active is useful for treating only asthma or the symptoms associated with asthma. The other actives can also have non-anti-asthma pharmacological properties in addition to their anti-asthma properties. For example, the other actives can have anti-cancer properties, or, alternatively, they can have no such anti-cancer properties.

As a further illustration, the Flt3 ligand can be administered in a composition which further includes a CpG nucleic acid, or the Flt3 ligand can be administered in a composition which is substantially free from CpG nucleic acids. As used herein, “CpG nucleic acid” is defined as in U.S. Pat. No. 6,429,199 to Krieg et al., which is incorporated by reference. For the purposes of the present invention, a composition is to be deemed as being “substantially free from CpG nucleic acids” if it contains no detectable amount of a CpG nucleic acid or if it contains CpG nucleic acid in an amount so low that the CpG nucleic acid is not pharmacologically effective.

As yet a further illustration, the Flt3 ligand can be administered in a composition which further includes a zalphall ligand antagonist, or the Flt3 ligand can be administered in a composition which is substantially free from zalphall ligand antagonists. As used herein, “zalphall ligand antagonists” is defined as in U.S. patent application Publication No. 20020137677 of Sprecher et al., which is incorporated by reference. For the purposes of the present invention, a composition is to be deemed as being “substantially free from zalphall ligand antagonists” if it contains no detectable amount of a zalphall ligand antagonist or if it contains zalphall ligand antagonist in an amount so low that the zalphall ligand antagonist is not pharmacologically effective.

The method of the present invention can optionally include other steps. For example, the method of the present invention can further include administering a pharmacologically active agent prior to, during, or subsequent to Flt3 ligand administration. Illustratively, the method of the present invention can further include the separate step of administering a bronchodilator, an HIV peptide vaccine, a CpG nucleic acid, or a zalphall ligand antagonist. Alternatively, the method of the present invention can be practiced such that it does not further include the step of administering bronchodilator, an HIV peptide vaccine, a CpG nucleic acid, or a zalphall ligand antagonist. As used herein, “HIV peptide vaccine” has the meaning set forth in Pisarev et al., “Flt3 Ligand Enhances the Immunogenicity of a gag-based HIV-1 Vaccine,” Int. J. Immunopharmacol., 22(11):865-876 (2000), which is incorporated by reference.

The method of the present invention can be carried out on any suitable subject. Suitable subjects include, for example mammals, such as rats, mice, cats, dogs, horses, monkeys, and humans. Suitable human subjects include, for example, those who suffer from cancer, those who do not suffer from cancer, those who suffer from colon cancer, those who do not suffer from colon cancer. Other suitable human subjects include, for example, those who suffer from asthma, those who do not suffer from asthma but who are exposed to allergens which cause asthma, and/or those who do not suffer from asthma but who are at elevated risk for developing asthma.

It will be appreciated that the actual preferred amount of Flt3 ligand to be administered according to the present invention will vary according to the particular compound, the particular composition formulated, and the mode of administration. Many factors that may modify the action of the Flt3 ligand (e.g., body weight, sex, diet, time of administration, route of administration, rate of excretion, condition of the subject, drug combinations, and reaction sensitivities and severities) can be taken into account by those skilled in the art. Administration can be carried out continuously or periodically within the maximum tolerated dose. Optimal administration rates for a given set of conditions can be ascertained by those skilled in the art using conventional dosage administration tests.

The present invention is further illustrated with the following examples.

### EXAMPLES

### Example 1

### Materials and Methods

Sensitization and treatment. Four- to five-week-old female Balb/c mice (Harlan Laboratories, Indianapolis, Ind.) were housed according to the NIH guidelines and were allowed constant access to food and water. Intraperitoneal (“i.p.”) injections of 6 mg of grade V chicken egg ovalbumin (“Ova”) (Sigma-Aldrich, St. Louis, Mo.) adsorbed to 2.25 mg (Imject Alum Pierce, Rockford, Ill.) and dissolved in PBS were delivered on days 0 and 8. Aerosol sensitization with 0.2% Ova for 10 min using an Ultra-Neb 90 nebulizer (DeVilbiss, Somerset, Pa.) was carried out on days 27, 28 and 29. Non-sensitized control mice received injection and aerosolization of PBS alone. Mice received i.p. injection of 6 μg of rhFlt-3 ligand (“FL”) in PBS (PeproTech, Rocky Hill, N.J.) on day -6 through day 6.

Pulmonary functions. On day 31, all mice were placed in whole-body plethysmograph chambers Buxco Electronics (Troy, N.Y.), and baseline Penh readings were taken. Penh is a dimensionless unit used to monitor airway resistance and has been positively correlated with pulmonary resistance R_{L} and change in intrapleural pressure ΔP as well as the response to Ova challenge. Challenge with aerosolized 0.2% Ova for 5 min was followed by recording of breathing parameters for 9 h to continuously record both the early allergic response (“EAR”) and late allergic response (“LAR”). The area-under-the-curve (“AUC”) was determined for each animal using the baseline and Penh values from 0 to 15 min for EAR and the baseline and Penh values from 1 to 6 h for LAR (GraphPad Prism ver. 3 statistical software, San Diego, Calif.). On day 32, 24 h following Ova challenge, mice were again placed within the chambers and challenged with increasing doses of methacholine (Sigma-Aldrich) to measure airway hyperresponsiveness (“AHR”). Penh values for each animal were expressed as a fold-increase from the baseline reading and referred to as Penh index.

Bronchoalveolar lavage. Bronchoalveolar lavage (“BAL”) fluid was retrieved from each animal via cannulation of the exposed trachea and gentle flushing of the lungs with four 1-ml aliquots of PBS. Aliquots were pooled for individual animals preceding centrifugation and separation of pelleted cells and supernatant. Cytospin preparations of BAL cells were stained using Hema 3 (Biochemical Sciences, Swedesboro, N.J.) differential stain and relative cell populations were determined using standard morphological criteria.

Cytokine assays. Cytokines were measured in the supernatants of BAL fluid. Antibody pairs for IL-4, IL-5 and IFN-γ as well as standards (both from Pharmingen, San Diego, Calif.) were used according to manufacturer's recommendations using streptavidin-horseradish peroxidase (“HRP”) and 3,3′, 5,5′-tetramethyl-benzidine (“TMB”) (Sigma-Aldrich) as a substrate. Assay sensitivities for IL-4, IL-5 and IFN-γ were 5, 5, and 10 pg/ml, respectively.

Serum immunoglobulin analysis. Blood collected after sacrifice on day 26 was immediately centrifuged, and serum was collected and stored at −70° C. until analysis. ELISA for both total and antigen-specific IgE was conducted as described in Hopfenspirger et al., “Mycobacterial Antigens Attenuate Late Phase Response, Airway Hyperresponsiveness, and Bronchoalveolar Lavage Eosinophilia in a Mouse Model of Bronchial Asthma,” Int. Immunopharmacol., 1(9-10):1743-1751 (2001) and Wilder et al., “Dissociation of Airway Hyperresponsiveness from Immunoglobulin E and Airway Eosinophilia in a Murine Model of Allergic Asthma,” Am. J. Respir. Cell. Mol. Biol., 20(6):1326-1334 (1999) (“Wilder”), which are hereby incorporated by reference. Both assays were developed with TMB substrate and read at 450 nm using a Bio-Rad microplate reader and software. Sensitivity for total IgE levels was 1 ng/ml. Antigen-specific IgE results have been expressed in units of absorbance (optical density)

Statistical analysis. Data were analyzed using GraphPad PRISM statistical analysis and graphing software. Unpaired Student's t-test was used to determine differences between the groups. A p value of ≦0.05 was considered significant.

### Example 2

### Results

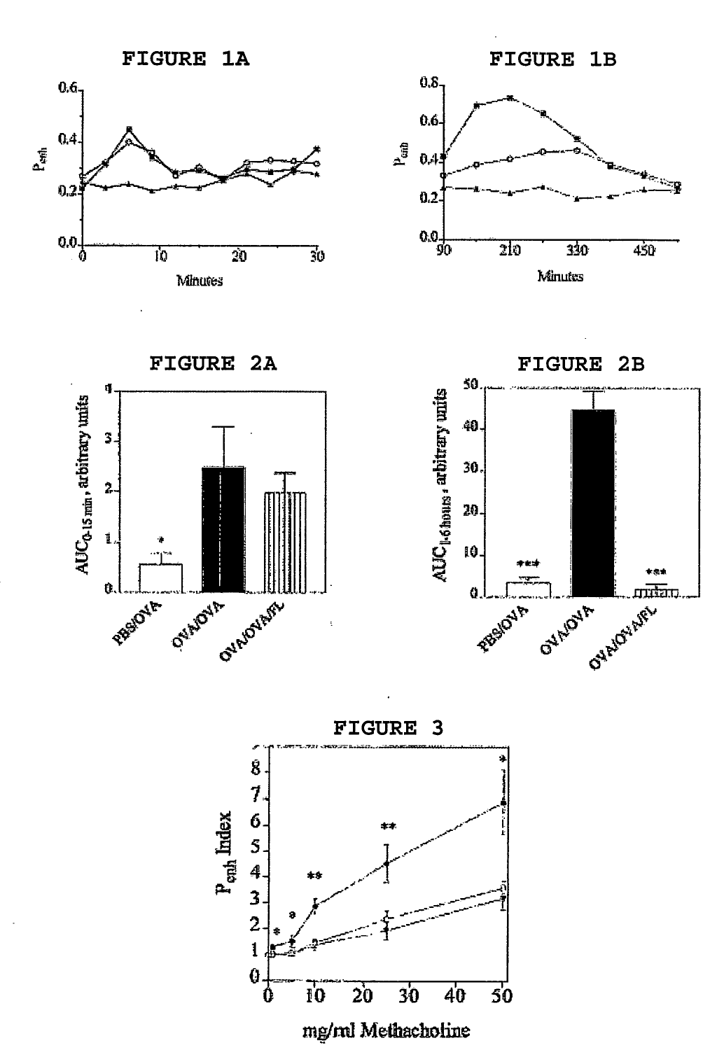

To determine whether FL could suppress asthma-like conditions in an Ova mouse model, we injected FL daily for 13 days, starting 6 days prior to sensitization. Previous studies by other investigators found significant increases in both peripheral and splenic white blood cells with FL treatment after 6-8 days, which roughly corresponds with the timing of sensitization day 0 in our study. Furthermore, these investigators observed a return to baseline values of these and other parameters 1 week after cessation of FL treatment, a time in our sensitization protocol that is about 1 week prior to aerosol Ova sensitization and challenge. Analysis of Ova-specific airway resistance of both EAR and LAR revealed that non-sensitized control mice had significantly lower airway resistance during either EAR or LAR (p≦0.05, EAR and p≦0.001, LAR; both compared to sensitized controls). FL-treated mice exhibited significant protection during LAR p≦0.001 but were without EAR protection. This is shown in FIGS. 1A, 1B, 2A, and 2B.

More particularly, FIGS. 1A and 1B show early allergic response (FIG. 1A) and late allergic response (FIG. 1B) in a non-sensitized control group (solid triangle), ovalbumin-sensitized and challenged (solid squares), and ovalbumin-sensitized, challenged and FL-treated (open circles) groups. Mice were challenged with 0.2% ovalbumin on day 31 and Penh values were recorded immediately thereafter every 3 min up to the first 30 min (FIG. 1A) followed by every 15 min up to 8 h (FIG. 1B) in individual barometric plethysmograph chambers. The data shown in FIGS. 1A and 1B are representative data from one animal in each group for the early and late allergic response.

FIGS. 2A and 2B show the effect of FL treatment on the EAR and LAR as calculated by the AUC. More particularly, two groups of mice were sensitized to Ova (plus one non-sensitized control group) and treated with either vehicle or FL, 6 μg/day/mouse for 13 days. In FIG. 2A, following challenge with ovalbumin, Penh values were recorded immediately thereafter, and AUC from 0 to 15 min was calculated for individual mice and expressed in arbitrary units. Non-sensitized control mice (open bar) exhibited significantly less airway resistance compared to sensitized controls (black bar), while treatment with FL failed to suppress this response (striped bar). In FIG. 2B, the pulmonary function in the same mice was examined using barometric plethysmography from 1 to 6 h. Penh units were recorded and calculated as AUC using the same individual baselines as used for EAR. Significant suppression of the airway resistance was observed in the non-sensitized control animals (open bar) as well as in the FL-treated controls (striped bar) compared to sensitized controls (black bar). The data shown in FIGS. 2A and 2B are shown as means±S.E.M. for six animals in each group with comparisons to sensitized control animals (*p≦0.05; ***p≦0.0001).

These data demonstrate that FL suppressed not only the maximal response during LAR (FIGS. 1A and 1B) but also the total LAR, as calculated by the AUC (FIGS. 2A and 2B). The finding that EAR was unaffected was further corroborated by the serum IgE analysis. Not only were mean serum total IgE concentrations unaffected by FL treatment, the values were increased, albeit insignificantly. This is shown in Table 1, where values are presented as means±S.E.M. for six animals per group (*p≦0.01).

TABLE 1

| | Total IgE | Ova-specific IgE |
|---|---|---|
| Study groups | (ng/ml) | (optical density) |
| Non-sensitized | ≦100 | 0 |
| Sensitized | 1907 ± 233* | 0.07 ± 0.02* |
| Sensitized/FL-treated | 2749 ± 430 | 0.05 ± 0.04 |

Table 1 also shows that there was no effect of FL on Ova-specific serum IgE, which represented about 6% of the total serum IgE in the antigen-sensitization protocol used in this study. Other investigators have shown that B cell progenitors, although responsive to FL, lose their capacity to react to FL early in maturation, just preceding the acquisition of CD24, a marker for Ig gene rearrangement. FL may be ineffective in directly influencing class-specific responses from B cells to antigen. Our results, which examined B cell (antigen presenting cell)/T cell interactions during sensitization, were unaffected by FL administration as IgE levels were unchanged.

Twenty-four hours after antigen challenge, the mice were challenged non-specifically with increasing doses of methacholine, and Penh values (later translated to the Penh index) were recorded by barometric plethysmography. In support of the suppressed LAR results, AHR to methacholine was also suppressed after FL treatment. This is demonstrated in FIG. 3, which is a graph showing the effect of FL treatment on AHR to methacholine. Groups of mice were sensitized to Ova (except the non-sensitized controls) and treated with either vehicle or FL, i.p. Twenty-four hours following Ova challenge, mice were placed into individual barometric plethysmograph chambers and exposed to increasing concentrations of aerosolized methacholine in PBS. Penh index refers to fold-increase over baseline Penh readings for each individual animal. FL-treated animals (open triangles) showed significant suppression of AHR at each dose of administered methacholine. The data are presented as means±S.E.M. for six animals in each group with comparisons to sensitized control (**p<0.01; *p<0.05). Although not universally accepted (Wilder, which is hereby incorporated by reference), the LAR and AHR results have been positively linked to the presence of eosinophils in the airways (Cielewicz et al., “The Late, But Not Early, Asthmatic Response is Dependent on IL-5 and Correlates with Eosinophil Infiltration,”J. Clin. Invest., 104:301-308 (1999), which is hereby incorporated by reference). Interleukin-5 is a critical growth factor for eosinophils and also acts as a potent stimulant and chemotactic factor (Teran, “Chemokines and IL-5: Major Players of Eosinophil Recruitment in Asthma,” Clin. Exp. Allergy, 29:287-290 (1999), which is hereby incorporated by reference). Indeed, we observed significantly decreased numbers of eosinophils in BAL fluid immediately following methacholine challenge. This is demonstrated in Table 2. More particularly, in these experiments, groups of mice were sensitized to Ova (except the non-sensitized controls) and treated with either PBS or FL. Twenty-four hours following Ova challenge and immediately following methacholine challenge, mice were sacrificed with a lethal injection of pentobarbital. Tracheas were surgically exposed, cannulated, and the lungs were flushed with PBS. Recovered cells were counted, and major populations were identified using standard morphological criteria. The data in Table 2 are presented as means±S.E.M. for six animals in each group (*p<0.01, compared to sensitized control; #p<0.01 for sensitized or sensitized/FL-treated vs. non-sensitized comparisons).

TABLE 2

| Study | Eosinophils | Macrophages | Lymphocytes | Neutrophils |
|---|---|---|---|---|
| groups | (×10³/ml) | (×10³/ml) | (×10³/ml) | (×10³/ml) |
| Non- | 0.0 ± 0.0 | 202 ± 66 | 32 ± 12 | 7.3 ± 3 |
| sensitized | | | | |
| Sensitized | 398 ± 53# | 480 ± 33# | 119 ± 13# | 68.9 ± 13# |
| Sensitized/ | 125 ± 36*# | 390 ± 41 | 151 ± 33# | 44 ± 14# |
| FL-treated | | | | |

A direct effect of FL activity on the Ova challenge is unlikely since the last injection of FL was given 14 days previously. Previous studies have shown that dendritic cells and stem cells return to normal within 7-8 days following the last injection of FL. Nonetheless, the induction of a type 1 T cell bias may be more long lasting. In addition, other researchers have found no direct effect of FL on eosinophil progenitors. We conclude that a lasting effect of the earlier FL exposure is responsible for the decrease in asthmatic symptoms and is likely to occur via an increased type 1 response.

BAL supernatants, collected immediately after methacholine challenge, were analyzed by ELISA for the presence of type 1 cytokine IFN-γ, and type 2 cytokines IL-4 and IL-5, which are central to the development of asthma. The data are presented in Table 3. More particularly, in these experiments, mice were sensitized and treated as described in Example 1. BAL was performed 24 h following Ova challenge, and supernatants were analyzed for cytokine levels with paired antibodies using ELISA. The data are presented as means±S.E.M. for six animals per group. There was a parallel change in the IL-5 and IFN-γ concentrations for the individual mice. Statistical significance was determined using Student's t test (#p<0.01 as compared to the non-sensitized group; *p<0.05; **p<0.01 compared to sensitized control).

TABLE 3

| | IL-4 (pg/ml) | IL-5 (pg/ml) | IFN-γ (pg/ml) |
|---|---|---|---|
| Non-sensitized | ≦10 | ≦5 | ≦10 |
| Sensitized | 90.3 ± 13.2# | 109.1 ± 9.8# | 79.4 ± 1.8# |
| Sensitized/FL- | 72.7 ± 8.2# | 49.3 ± 11.2*# | 177 ± 16.1**# |
| treated | | | |

Retention of significantly increased IL-4 levels supported the maintenance of Ova-specific airway resistance and serum IgE concentrations. In contrast, there was a significant reduction in IL-5 levels, which corroborated the reduced BAL fluid eosinophilia (Table 3). Finally, IFN-γ concentrations, a marker for type 1 T cell responses, were significantly increased in FL-treated animals compared to both groups (Table 3). Interestingly, there was a parallel change in IL-5 and IFN-γ concentrations for the individual mice. These responses were observed in samples collected 26 days after cessation of FL treatment, supporting a stable shift in the type 1/type 2 cytokine profile to a type 1 profile.

### Example 3

### Effect of FL on the Effects of Asthma in Established Asthmatic Mice

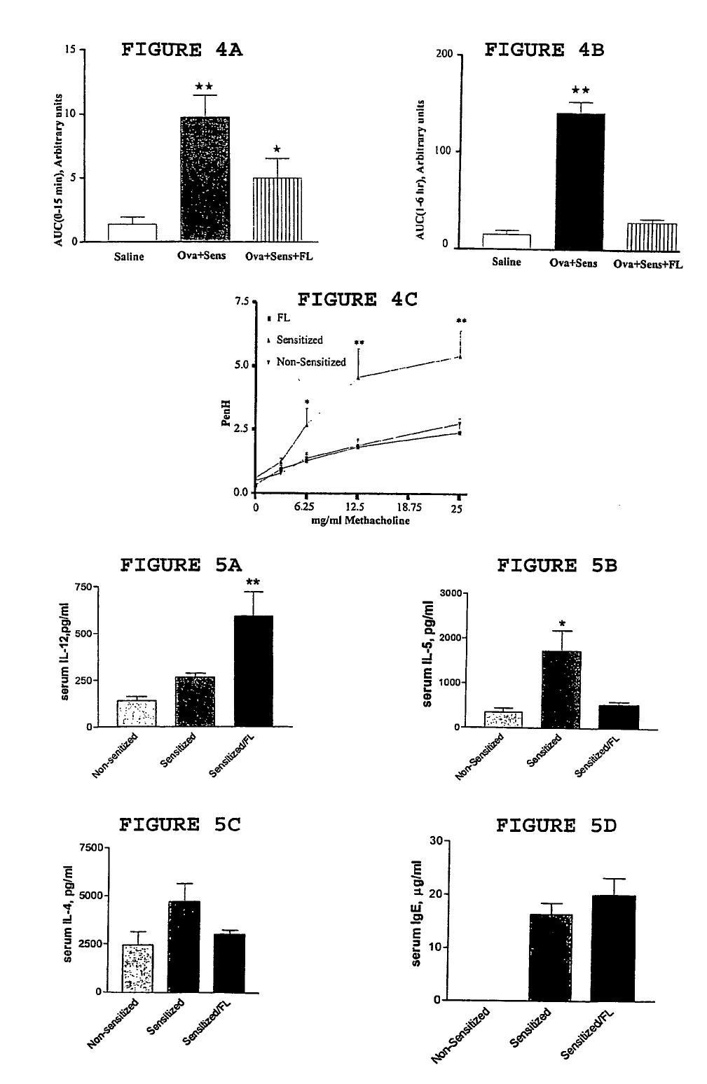

In order to examine the reversal effect of FL, we first sensitized and challenged mice with OVA. After the establishment of LAR and AHR to methacholine, mice were randomly divided in two groups: one group received pyrogen-free saline and the other received FL (300 μg/kg daily for 10 days). Two days after the final administration of FL, mice were challenged with OVA to examine EAR, LAR, and AHR to methacholine. The results are shown in FIG. 4A (EAR), FIG. 4B (LAR), and FIG. 4C (AHR). These data suggest that FL can attenuate LAR and abolish AHR to methacholine. Interestingly, in contrast to the preventive data shown in FIGS. 2A and 2B, the reversal experiments revealed a significant protection in the EAR following FL treatment. Treatment with FL alone in non-sensitized animals had no significant effect on pulmonary functions (data not shown).

### Example 4

### Effect of FL Treatment on Serum Cytokines and Serum Total IgE on Ovalbumin Pre-Sensitized and Challenged Mice

Experiments were carried out to examine the serum concentrations of IL-4, IL-5, IL-12, and total IgE in already established model of allergic airway inflammation. The results are presented in FIGS. 5A, 5B, 5C, and 5D. More particularly, Balb/c mice were sensitized and challenged with ovalbumin, and airway hyperresponsiveness to methacholine was established on day 31. Starting day 33 mice were administered with FL (6 μg, i.p.) daily for 10 days. On day 42 and 43, after recording pulmonary functions for EAR, LAR, and AHR, blood was collected to measure serum IL-12 (FIG. 5A), serum IL-5 (FIG. 5B), serum IL-4 (FIG. 5C), and serum total IgE concentration (FIG. 5D). Data is shown as mean±S.E.M. (n=6-8) (*p<0.05, **p<0.01). As the results show, FL significantly increased serum IL-12 levels and significantly decreased serum IL-5 levels. However, there was no effect of FL on serum IL-4 and serum total IgE concentration.

### Example 5

### Effect of FL Administration on Lung Histology in Antigen Pre-Sensitized and Challenged Mice

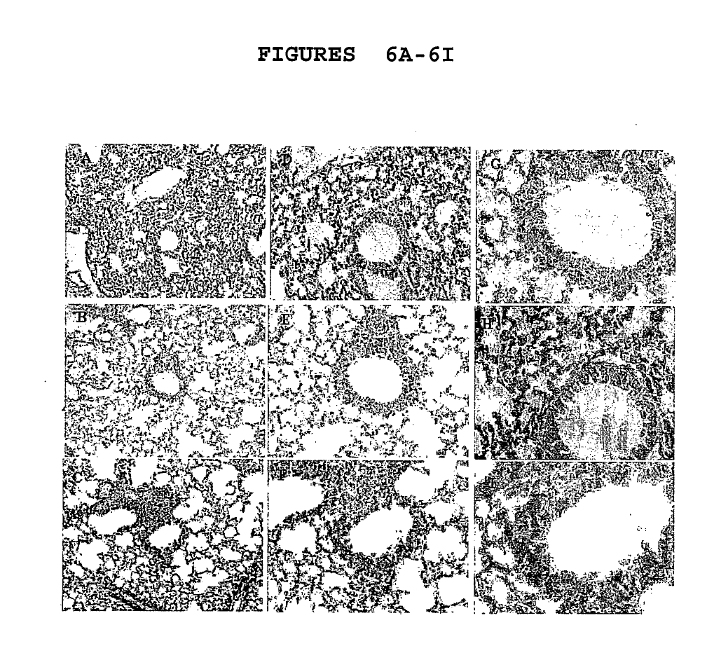

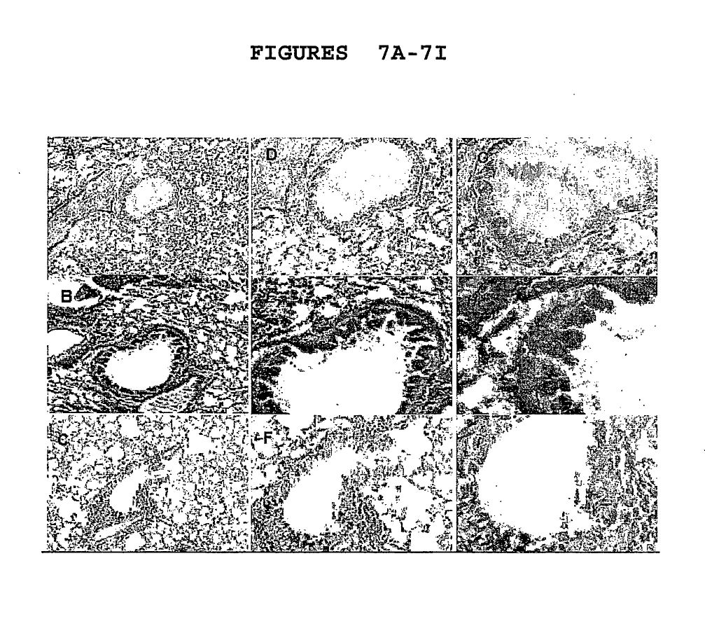

Experiments were carried out to examine the lung histology using H&E stain in the thin sections of lung from PBS-treated, OVA-sensitized and challenged, and OVA-sensitized and challenged mice followed by treatment with FL. The results are presented in FIGS. 6A-6I. More particularly, after sensitization and treatment, the mice were sacrificed, and the lungs were immediately collected and fixed in 10% buffered formalin. Thin sections (8 μm) were cut, stained with hematoxylin and eosin, and examined under light microscopy at 10× magnification (FIGS. 6A-6C), 20× magnification (FIGS. 6D-6F), and 40× magnification (FIGS. 6G-6I). The photomicrographs presented in FIGS. 6A-6I are representative of the observed lung histology of PBS-treated mice (FIGS. 6A, 6D, and 6G), Ova-sensitized and challenged mice (“the OVA group”) (FIGS. 6B, 6E, and 6H), and Ova-sensitized and challenged mice treated with FL for 10 days (“the OVA/FL group”) (FIGS. 6C, 6F, and 6I). In the OVA group, a massive peribronchial infiltration with eosinophils, thickening of the basement membrane, and de-epithelialization were seen (FIG. 6H). In contrast, after treatment with FL, an intact bronchial epithelial layer and no eosinophil infiltration were seen (FIGS. 6F and 6I), and the histology was comparable to PBS-treated group.

### Example 6

### Effect of FL Administration on Goblet Cell Hyperplasia and Mucus Hypersecretion in Allergen Pre-Sensitized and Challenged Mice

Experiments were carried out to examine the effect of FL treatment on mucus accumulation in the lower airways of PBS-treated, OVA-sensitized and challenged, and OVA-sensitized and challenged mice followed by treatment with FL. The results are presented in FIGS. 7A-7I. More particularly, after sensitization and treatment, the mice were sacrificed, and the lungs were immediately collected and fixed in 10% buffered formalin. Thin sections (8 μm) were cut, stained with periodic acid-Schiff reagent (“PAS”), and examined under light microscopy at 10× magnification (FIGS. 7A-7C), 20× magnification (FIGS. 7D-7F), and 40× magnification (FIGS. 7G-7I). The photomicrographs presented in FIGS. 7A-7I are representative of the PAS staining observed in the lungs of PBS-treated mice (FIGS. 7A, 7D, and 7G), Ova-sensitized and challenged mice (“the OVA group”) (FIGS. 7B, 7E, and 7H), and Ova-sensitized and challenged mice treated with FL for 10 days (“the OVA/FL group”) (FIGS. 7C, 7F, and 7I). The mucosubstances are stained in magenta by the PAS reaction and appear as dark areas in FIGS. 7A-7I. In the OVA group, there was a strong staining to PAS (FIGS. 7B, 7E, and 7H), which was significantly reduced by FL treatment (FIGS. 7C, 7F, and 7I).

### Example 7

### Effect of FL Administration on AHR to Methacholine by Whole Body Plethysmography in Unrestrained and Tracheostomized Mice

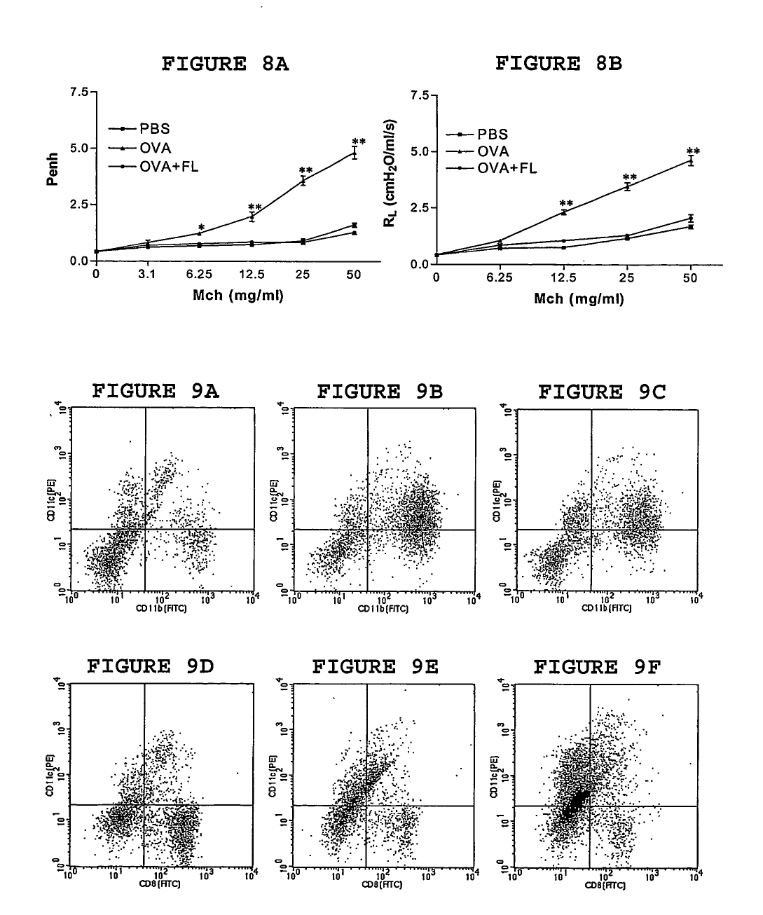

Experiments were carried out to compare AHR to methacholine (“Mch”) by two separate methods: (i) whole body plethysmography measuring enhanced Pause (Penh) in unrestrained free moving conscious mice, and (ii) in tracheostomized mice. Mice were sensitized and challenged with Ova. Initial AHR was established (on day 31), and the reversal effect of FL was examined by administration of 6 μg/d FL for 10 days, i.p. AHR to Mch was again measured one day after the last FL dose (day 42) in Penh values in unrestrained mice. The following day (day 43), the same animals were used to measure AHR to Mch in tracheostomized mice. In the latter method, mice were anesthetized with pentobarbital (25 mg/kg, i.p.) and ketamine (25 mg/kg, i.p.) and then paralyzed with pancuronium bromide (0.3 mg/kg, i.p.). Anesthesia and paralysis were maintained by supplemental administration of 10% of the initial dose every hour. After tracheostomy, an endotracheal metal tube was inserted in the trachea, and the animals were mechanically ventilated with tidal volume of 10 ml/kg and frequencies of 2.5 Hz. Oxygen was continuously supplied to the ventilatory system. After opening the thorax by midline sternotomy, end expiratory pressure of 2 cm H₂O was applied by placing the expired line underwater. A heating pad was used to maintain the body temperature. Tracheal pressure was measured by a piezoresistive microtransducer placed in the lateral port of the tracheal cannula. Tracheal flow was measured by means of a Fleisch pneumotachograph. All signals were amplified, filtered at a cutoff frequency of 100 Hz, and converted from analog to digital. The signals were sampled at a rate of 200 Hz and stored. In order to measure AHR to Mch, two deep inhalations (3 times tidal volume) were delivered to standardize volume history. Animals were challenged with saline aerosol delivered through the inspiratory line into the trachea. After baseline measurements, several doses of Mch aerosol were administered for 2 min in a dose-response manner (3.1 to 50 mg/ml). AHR to Mch was assessed using the changes in pulmonary resistance compared to baseline values. Data from six animals in each experimental group is shown below in FIG. 8A (unrestrained mice) and in FIG. 8B (anesthetized and tracheostomized mice). The data presented in FIGS. 8A and 8B are shown as mean±S.E.M. (n=6 in each group) (*p<0.05; **p<0.01). These data demonstrate that FL is effective in reversing AHR to Mch in Ova-sensitized and challenged mice.

### Example 8

### Study of the Phenotype and Function of Lung Dendritic Cells in FL-Treated Mice

Dendritic cells (“DCs”) were isolated, using MACS CD11c microbeads, from the lungs of same animals that were used for the measurement of AHR to Mch in Example 7. After removing the blood, lungs were excised, washed with PBS, and digested with collagenase D in HEPES media. CD11c+ cells were isolated by positive selection. Rat antimouse FcgR mAbs (1:200) were used to block FcgR non-specific binding. Cells were stained with anti CD11c-PE (1:200), anti CD11b-FITC (1:200), and anti CD8a FITC (1:200). Anti CD45R-FITC (1:400) was used to localize lymphocytes. The cell profiles were gated on DCs (based on high forward and 90° light scatter). Majority of lymphocytes and debris were gated out based on light scatter. Representative data from the three experimental groups are shown in FIGS. 9A-9F. More particularly, FIGS. 9A-9F are flow cytometry scatter plots showing the fluorescence characteristics of lung DCs in non-sensitized mice (PBS) (FIGS. 9A and 9D), in Ova-sensitized and challenged mice (OVA) (FIGS. 9B and 9E), and in Ova-sensitized and challenged mice treated with FL (OVA+FL) (FIGS. 9C and 9F). Cells were labeled with PE-conjugated CD11c+ antibody and with either FITC-conjugated CD11b antibody (FIGS. 9A-9C) or with FITC-conjugated CD8A antibody (FIGS. 9D-9F).

Cumulative data from these experiments are shown in Table 4 and are presented as mean±S.E.M. from at least 4 separate animals in each group.

TABLE 4

| Cell | | | |
|---|---|---|---|
| Phenotype | PBS | OVA | OVA + FL |
| CD11c + CD11b− | 11.3% ± 0.5% | 4.6% ± 0.1% | 10.4% ± 0.7% |
| CD11c + CD11b+ | 11.2% ± 0.6% | 46.2% ± 3.3% | 41.1% ± 1.7% |
| CD11c + CD8α+ | 8.2% ± 0.2% | 5.1% ± 0.7% | 4.8% ± 0.2% |
| CD11c + CD8α− | 8.9% ± 0.5% | 14.2% ± 0.6% | 21.6% ± 2.7% |

These experiments reveal a significant increase in CD11c+CD11b+ cells in OVA-sensitized and challenged lungs (46.2±3.3%) as compared to PBS (11.2±0.6%). CD8a+ cells did not increase in OVA group. Interestingly, FL did not show any effect on either CD11c+CD11b+ cells or CD11c+CD8a+ cells.

### Example 9

### Effect of FL Administration on Th2 Cytokine Production in Allergen Pre-Sensitized and Challenged Mice

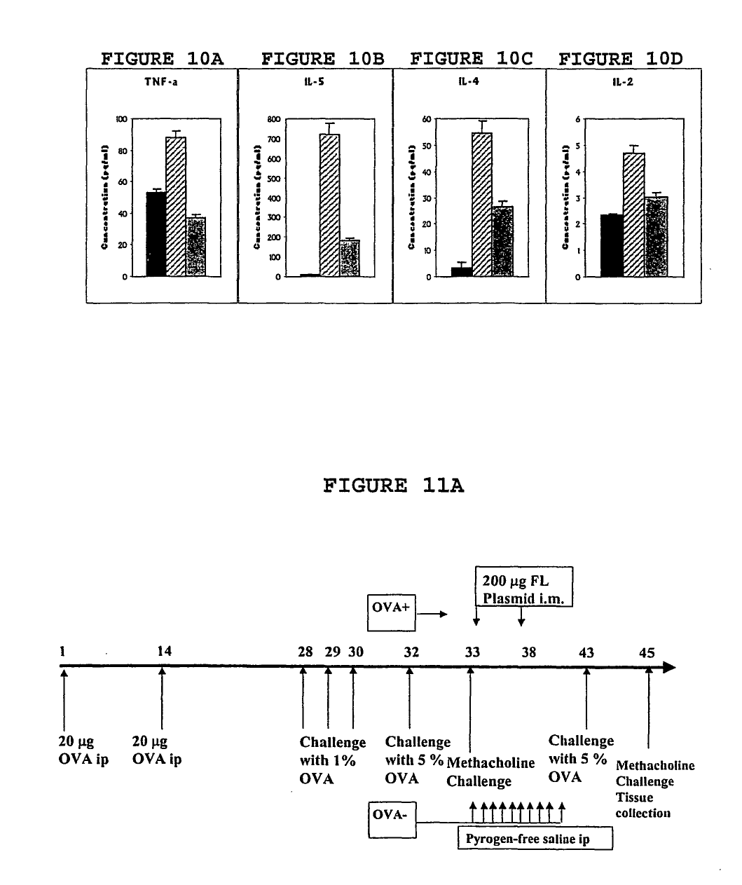

Bronchoalveolar lavage fluid (“BALF”) was collected from the animals in the experiments described in Example 7 by 2 washings of the lungs with a total of 1 ml PBS. Recovery of the BALF (0.7 ml) in various groups was same. BALF was centrifuged to remove cells, and the supernatant was immediately frozen for the measurement of cytokines. Cytokines (TNFα, IL-2, IL-4, IL-5, and IFN-γ) were measured by flow cytometric analysis using mouse Th1/Th2 cytokine Cytometric Bead Array (“CBA”) kit (BD Biosciences/Pharmingen). Data from six individual animals in each group are shown in FIG. 10A (TNFα), FIG. 10B (IL-5), FIG. 10C (IL-4), and FIG. 10D (IL-2). In each of FIGS. 10A-10D, the left, center, and right bars represent data from mice receiving PBS, Ova, and Ova+FL, respectively. These data show a significant increase in TNFα, IL-2, IL-4, and IL-5 concentrations in Ova-sensitized and challenged mice and a generalized decrease in all these cytokines in the BALF of FL-treated mice, the most stroking effect of FL treatment being observed on the levels IL-4 and IL-5. The level of IFN-γ in the BALF was less than the detectable range, and these data are not shown.

### Example 10

### Preparation and Use of FL as a Vaccine in the Treatment of Bronchial Asthma

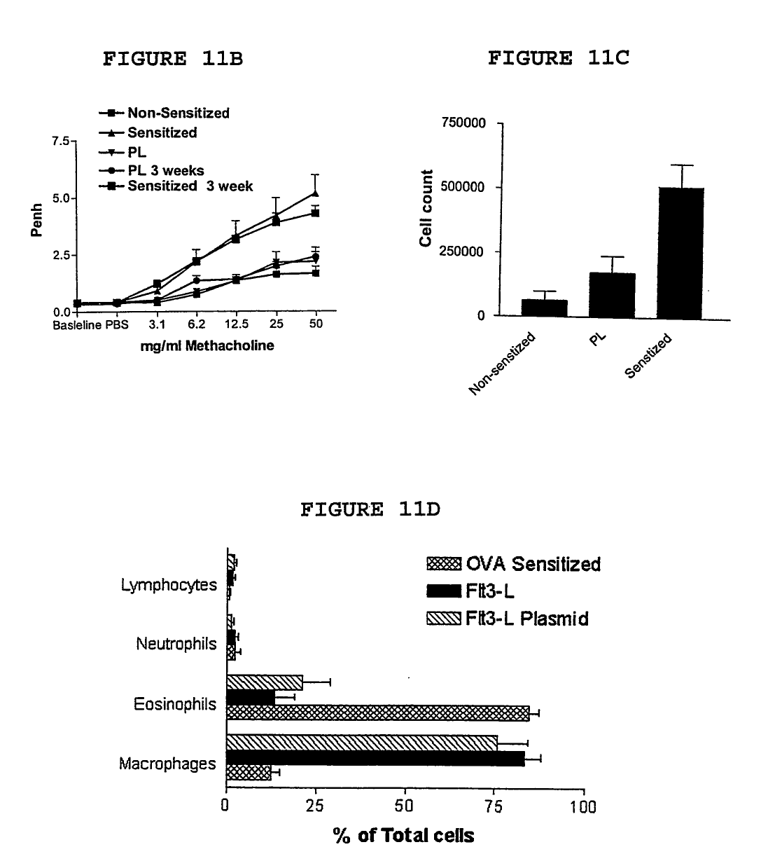

Plasmid pUMVC3-hFLex was obtained from Vector Core at the University of Michigan, Ann Arbor, Mich.) and was propagated in E. coli and purified. The resulting plasmid (“Plasmid FL”) was then administered to sensitized and challenged mice in accordance with the timeline presented in FIG. 11A. The numbers directly above the horizontal arrow represent days, day 1 being at the far left.

AHR to Mch was measured (using the protocol described above) in the non-sensitized mice, in the Ova-sensitized mice, in the Ova-sensitized and challenged mice treated with Plasmid FL (PL). The results are presented in FIG. 11B.

Bronchoalveolar lavage fluid (“BALF”) was collected from the non-sensitized mice, from the Ova-sensitized mice, and from the Ova-sensitized and challenged mice treated with Plasmid FL (PL). The number of cells in the BALFs was counted, and the results of this experiment are presented in FIG. 11C.

BALF was collected from the non-sensitized ice, from the Ova-sensitized mice, from the Ova-sensitized and challenged mice treated with Plasmid FL, and from Ova-sensitized and challenged mice treated with Flt3 ligand. The number of lymphocytes, neutrophils, eosinophils, and macrophages in the BALF from each group were counted, and the results are presented in FIG. 11D as percent of total cells in the BALF (N=6−8 per experimental group). There was a significant increase in BALF eosinophils in the Ova-sensitized and challenged mice, and, as FIG. 11D shows, both Plasmid FL and Flt3 ligand attenuated the increase in BALF eosinophilia.

The results set forth in FIGS. 11B, 11C, and 11D demonstrate that Plasmid FL suppresses airway hyperresponsiveness, limits infiltration of inflammatory cells into the airways, and lowers eosinophil levels.

Although preferred embodiments have been depicted and described in detail herein, it will be apparent to those skilled in the relevant art that various modifications, additions, substitutions and the like can be made without departing from the spirit of the invention and these are therefore considered to be within the scope of the invention as defined in the claims which follow.

1. A method of partially or completely preventing or reversing the effects of asthma in a subject, said method comprising:
administering to the subject an effective amount of a Flt3 ligand.

2. A method according to claim 1, wherein the Flt3 ligand is administered by administering, to the subject, a pharmaceutically acceptable composition which comprises Flt3 ligand and a pharmaceutically acceptable carrier.

3. A method according to claim 2, wherein the pharmaceutically acceptable composition is substantially free from CpG nucleic acids.

4. A method according to claim 2, wherein the pharmaceutically acceptable composition is substantially free from zalphall ligand antagonists.

5. A method according to claim 1, wherein the Flt3 ligand is administered by administering, to the subject, a nucleic acid molecule encoding a Flt3 ligand under conditions effective to permit the nucleic acid molecule to express the Flt3 ligand in the subject.

6. A method according to claim 5, wherein the nucleic acid molecule is administered in the form of a plasmid or viral vector.

7. A method according to claim 1, wherein the Flt3 ligand is administered by administering, to the subject, a conjugate which comprises Flt3 ligand and an allergen.

8. A method according to claim 7, wherein the conjugate comprises Flt3 ligand and an allergen which are coupled to one another by a covalent, hydrogen, and/or ionic bond.

9. A method according to claim 1, wherein the Flt3 ligand is administered intranasally.

10. A method according to claim 1, wherein the Flt3 ligand is administered in the form of an aerosol.

11. A method according to claim 1, wherein the Flt3 ligand is administered intraperitoneally.

12. A method according to claim 1, wherein the Flt3 ligand is recombinant Flt3 ligand.

13. A method according to claim 1, wherein the subject suffers from asthma and wherein the Flt3 ligand is administered to the subject under conditions effective to reverse the effects of asthma.

14. A method according to claim 1, wherein the subject does not suffer from asthma but is exposed to allergens which cause asthma and wherein the Flt3 ligand is administered to the subject under conditions effective to prevent the development of asthma.

15. A method according to claim 1, wherein said method does not further comprise administering an HIV peptide vaccine.

16. A method according to claim 1, wherein the subject does not suffer from colon cancer.

17. A method according to claim 1, wherein the subject does not suffer from cancer.

18. A composition comprising a Flt3 ligand and a pharmaceutically acceptable aerosolizing agent.

19. A conjugate comprising a Flt3 ligand and an allergen.
